(19) United States
(12) Patent Application Publication
(10) Pub. No.: US 2014/0214138 A1
(43) Pub. Date: Jul. 31, 2014

(54) HYBRID MEDICAL COOLING PAD WITH INCREASED WATER FLOW AREA

(71) Applicant: Medivance Incorporated, Louisville, CO (US)

(72) Inventors: Marc E. Voorhees, Arvada, CO (US); Owen Mandry, Longmont, CO (US)

(73) Assignee: Medivance Incorporated, Louisville, CO (US)

(21) Appl. No.: 13/969,025

(22) Filed: Aug. 16, 2013

Related U.S. Application Data
(60) Provisional application No. 61/758,033, filed on Jan. 29, 2013.

(51) Int. Cl. A61F 7/10

(52) U.S. Cl. CPC A61F 7/10
USPC 607/104

(57) ABSTRACT

A medical pad having a dual layer portion including a fluid circulation and containment layers and a single layer portion including the fluid circulation layer. The fluid circulation layer is for containing a first thermal-exchange fluid circulatable therethrough, with the medical pad being operable for thermal exchange between the first thermal-exchange fluid and a patient through a first side of the fluid circulation layer. A fluid containment layer of the medical pad is interconnected to a portion of second side of the fluid circulation layer, opposite to the first side of the fluid circulation layer. The fluid containment layer encloses a second thermal-exchange fluid that may have a freezing point of 0° C. or less. Portions of the fluid circulation layer that extend beyond the fluid containment layer define flaps that provide additional area for fluid circulation.

### RELATED APPLICATIONS

This application claims priority to U.S. Provisional Patent Application No. 61/758,033 filed Jan. 29, 2013, entitled “HYBRID MEDICAL COOLING PAD WITH INCREASED WATER FLOW AREA,” which application is incorporated herein by reference in its entirety.

### FIELD OF THE INVENTION

The present invention relates to cooling medical patients. More specifically, this application relates to a cooling pad for treating medical patients benefiting from cooling treatment and to methods for using such a cooling pad.

### BACKGROUND OF THE INVENTION

There are a number of medical conditions in which systemic cooling is an effective therapy. For example, rapid systemic cooling of stroke and head-trauma patients has significant therapeutic benefits. Stroke is a major cause of death and neurological disability, but recent research has suggested that even though a stroke victim's brain cells may lose their ability to function during the stroke, they do not necessarily die quickly. Brain damage resulting from a stroke may take hours to reach maximum effect. Neurological damage may be limited and the stroke victim's outcome improved if a cooling neuroprotectant therapy is applied during that timeframe.

Similar possibilities exist with the victims of trauma such as may result from vehicle crashes, falls, and the like. Such trauma may cause brain injury through mechanisms that have overlap with elements in the genesis of neurologic damage in stroke victims. Delayed secondary injury at the cellular level after the initial head trauma event is recognized as a major contributing factor to the ultimate tissue loss that occurs after brain injury.

Cooling therapy has been shown in a number of studies to confer neuroprotection in stroke victims and may hasten neurologic recovery. Such cooling therapy may be applied with the use of a medical cooling pad that is placed on the patient. For example, the pad might be placed on the patient's torso and fluid such as water or air circulated through the pad. Thermal energy is then exchanged between the patient and the circulated fluid so that when the temperature of the fluid is lower than the desired temperature of the patient, the patient is cooled.

### SUMMARY OF THE INVENTION

Embodiments of the invention provide a medical pad that comprises a plurality of layers. A first layer or fluid circulation layer of the medical pad is for containing a first thermal-exchange fluid circulatable therethrough (e.g., cooled fluid circulated via an interconnected pump/heat exchange unit). The fluid circulation layer includes at least one fluid path or fluid channel disposed between first and second surfaces. The medical pad is selectively positionable to contact a patient on the first surface thereof, and is operable for thermal exchange between the circulatable first thermal-exchange fluid and a patient through a first side or surface of the fluid circulation layer and the first side or surface of the medical pad. A second layer or fluid containment layer of the medical pad is disposed over a portion of less than all of a second surface of the fluid circulation layer, opposite to the first surface of the fluid circulation layer. The fluid containment layer encloses a second thermal-exchange fluid between first and second surfaces. One or more portions of the fluid circulation layer that is not covered on the second surface by the fluid containment layer extend beyond one or more lateral edges of the fluid containment layer and define flap portions. In this regard, the medical pad has a dual layer portion having both fluid circulation and fluid containment layers and one or more flap portions having a fluid circulation layer.

In one arrangement, the one or more flap portions may have an area or combined area that is at least equal to an area of the portion of the fluid circulation layer covered by the fluid containment layer. In further arrangements, the flap portion(s) may have an area that is greater than the area of the dual layer portion of the medical pad. In further arrangements, the flap portion(s) may have an area that is 120%, 140%, 160%, 180%, 200% or greater than the area of the dual layer portion of the medical pad.

An adhesive surface may be disposed on the first surface of the fluid circulation layer and adapted for releasable adhesive contact with skin of a patient. In certain embodiments, the adhesive surface extends across at least a majority of a lateral extent of the fluid circulation layer. One or more release liners may be disposed over the adhesive surface. For example, one release liner may be disposed over the first surface of the fluid circulation layer opposite to the second surface of the fluid circulation layer covered by the fluid containment layer. Likewise, each flap portion of the fluid circulation layer that extends beyond the fluid containment layer may have a separate release liner.

The medical pad is operable for thermal exchange between the second thermal-exchange fluid and the patient through the first surface of the medical pad. In some approaches the second thermal-exchange fluid may comprise a liquid having a freezing point of 0° C. or less. In turn, in such approaches, the second thermal-exchange fluid contained in the fluid containment layer may be chilled, e.g., to at least a semi-frozen state, prior to use. Additionally, in such approaches, the second thermal-exchange fluid may comprise liquid in a gel form. For example, a gel material comprising a water/polymer matrix may be utilized. In some implementations, shape-holding gels may be utilized.

The medical pad may be configured for different levels of thermal communication with the first and second thermal-exchange fluids in different embodiments. In some embodiments, for example, greater than 30% of the dual layer portion of the medical pad in contact with the patient is in thermal communication with the first thermal-exchange fluid (e.g., located adjacent thereto), and in a specific embodiment, approximately 50% of the area of the dual layer portion of the medical pad in contact with the patient is in thermal communication with the first thermal-exchange fluid (e.g., located adjacent thereto). Similarly, in other embodiments, greater than 30% of an area of the dual layer portion of the medical pad in contact with the patient is in thermal communication with the second thermal-exchange fluid (e.g., located adjacent thereto), and in a specific embodiment, approximately 50% of the area of the dual layer portion of the medical pad in contact with the patient is in thermal communication with the second thermal-exchange fluid (e.g., located adjacent thereto). In one embodiment, approximately 50% of the area of the dual layer portion of the medical pad in contact with the patient is in thermal communication with the first thermal-exchange fluid (e.g., located adjacent thereof) and approximately 50% of the area of the medical pad in contact with the patient is in thermal communication with the second thermal-exchange fluid (e.g., located adjacent thereto).

The flap portions of the medical pad may be configured to have different levels of thermal communication with the first thermal-exchange fluid relative to the dual layer portion of the medical pad. For instance, the flap portions may have greater than 80% of their area in contact with the first thermal-exchange fluid while the dual layer portion has approximately 50% of its area in contact with the first thermal exchange fluid. Multiple other combinations are possible and within the scope of the invention.

The fluid circulation layer typically includes at least a first plurality of fluid channels. In one arrangement, the first plurality of fluid channels are adjacent and have first coincidental configurations. The provision of multiple channels of coincidental configurations may facilitate the maintenance of a desired thermal gradient across the pad-to-patient interface, e.g., since any patient pressure occlusion within the fluid containing layer can be localized and fluid flow shunting can be minimized.

In one arrangement, the first plurality of fluid channels may have coincidental, serpentine configurations. Further, the pad may include a second plurality of adjacent fluid channels within the fluid circulation layer. Such a second plurality of fluid channels may have second coincidental configurations different than the coincidental, serpentine configurations of the first plurality of fluid channels. The provision of at least two different sets of fluid channels having corresponding coincidental configurations which are different enhances the ability to adapt the pad to conform to bodily portions of differing complex configurations, while also providing for a highly reliable and efficient degree of thermal exchange with a patient.

In one arrangement, the first plurality of fluid channels are disposed in the dual layer portion of the medical pad and the second plurality of fluid channels are disposed in the flap portion(s) of the medical pad. In conjunction with this arrangement, the medical pad may further include one or more intermediate fluid staging chambers for receiving fluid from one of the first and second plurality of fluid channels and distributing such fluid into the other of the first and second plurality of fluid channels.

In an additional arrangement, the medical pad may include a dual layer portion and first and/or second flap portions that are separately and pivotably interconnected to the dual layer portion. In one arrangement, each flap portion is pivotable about a pivot axis that is transverse to a lateral edge of the medical pad (e.g., at an angle of between about 70° to 110°). Further, the first plurality of fluid channels may be disposed so that each of the channels include a U-shaped portion located in one of the first and second flap portions. Such segmentation and channeling features further facilitate the ability to achieve conformal positioning of the inventive pad on bodily portions having differing complex configurations.

The fluid containment layer may comprise a plurality of chambers. In some such embodiments, the plurality of chambers may each enclose a corresponding different portion of the second thermal-exchange fluid therewithin. In some embodiments, at least a portion of each of the plurality of enclosed chambers may be located laterally adjacent (e.g., side-by-side) with corresponding first thermal-exchange fluid containment portions, e.g., fluid flow channels, of the fluid circulation layer.

Each of the plurality of chambers may project away from the second surface of the fluid circulation layer with indentations defined therebetween. For instance, the plurality of chambers may, in one embodiment, define a waffle-shaped configuration. The provision of indentations between adjacent chambers (e.g., laterally and/or longitudinally extending indentations), together with the utilization of pliable materials to define the first and second layers, allows for a degree of pivotal, or hinge-like movement, about such indentations. Such feature facilitates medical contact with a patient and is particularly advantageous when the second thermal-exchange fluid is in a solid or semi-solid state (e.g., ice).

Ports may be fluidly interconnected to the fluid circulation layer for selective interconnection to a separate pump/heat exchanger unit provided for circulation of the first thermal-exchange fluid. In such cases, a first port is fluidly interconnected to the fluid circulation layer for circulating the first thermal-exchange fluid into the fluid circulation layer and a second port is fluidly interconnected to the fluid circulation layer for circulating the first thermal-exchange fluid out of the fluid circulation layer.

In a further arrangement, one or more additional layers may be applied to the medical pad. In one arrangement, a top or insulative layer may be disposed over at least a portion of the top surface of the fluid containment layer. In a further arrangement, the top layer may further include a plurality of corrugations that allow the medical pad to expand or collapse when applied to a non-planar surface (e.g., when the adjacent chambers experience pivotal or hinge-like movement). For example, the top layer may expand or collapse in an accordion-like manner. In a yet further arrangement, the top layer may include one or more recesses that extend across a lateral extent thereof in one or more directions. These recesses may extend below a top surface of the top layer and/or be disposed within the indentations between adjacent chambers of the underlying fluid containment layer.

Embodiments of the invention may also comprise different thermal properties for the thermal-exchange fluids. For example, at least one of the first thermal-exchange fluid or the second thermal-exchange fluid may have a thermal conductivity that exceeds 5.0 W/mK, that exceeds 10.0 W/mK, that exceeds 50.0 W/mK, that exceeds 100.0 W/mK, or that exceeds 250 W/mK in various embodiments. The at least one of the first thermal-exchange fluid or the second thermal-exchange fluid may comprise a liquid containing a material having a thermal conductivity that exceeds a thermal conductivity of the liquid by at least a factor of 10, a factor of 50, a factor of 100, a factor of 500, or a factor of 1000 in various embodiments.

Embodiments of the invention also include methods for contact cooling of a patient and for providing a medical pad for contact cooling. In the former aspect, a medical pad may be positioned on a patient such that at least a dual layer portion of the medical pad having a fluid circulation layer and a fluid containment layer contacts the patient. Thermal energy is transferred as part of a first transferring step between a fluid containment layer of the medical pad and the patient. The fluid containment layer may enclose a first thermal-exchange fluid that is chilled, e.g., to a temperature of 5° C. or less (e.g., frozen water). Thermal energy is also transferred as part of a second transferring step between a circulation layer of the medical pad and the patient by circulating a second thermal-exchange fluid through the circulation layer of the medical pad. Further, the second transferring step may include circulation the second thermal-exchange fluid through the dual layer portion of the medical pad and one or more single layer portions of the medical pad.

The first transferring step may be performed over greater than 30% of an area of the dual layer portion of the medical pad in contact with the patient, and in some cases is performed over approximately 50% of an area of the medical pad in contact with the patient. Similarly, the second transferring step may be performed over greater than 30% of an area of the dual layer portion of the medical pad in contact with the patient, and in some cases is performed over approximately 50% of the area. In addition, the second transferring step may be performed over one or more single layer portions of the medical pad.

The first and second transferring steps may be at least partially offset. For instance, the first transferring step may be initiated at a first location and the second transferring step may be initiated at a second location different from the first location. In such cases, the patient may be moved from the first location to the second location between initiation of the first transferring step and initiation of the second transferring step, such as in an ambulatory vehicle. In some embodiments, at least a portion of the first transferring step is completed during the moving step.

The method may also comprise cooling the medical pad prior to each of the positioning, first transferring, and second transferring steps. In such cases, the first thermal-exchange fluid may be chilled by such cooling to a temperature below at least 5° C. In some approaches, the first thermal-exchange fluid may be chilled to a frozen or semi-frozen state prior to positioning at the pad on a patient.

In some embodiments, the medical pad may be positioned on the patient by adhering the medical pad to skin of a bodily portion of the patient. In such embodiments, one or more liners may be removed from an adhesive surface of the medical pad, and the adhesive surface of the medical pad may be contacted with the skin of the bodily portion of the patient. The adhesive surface may extend across at least a majority of a lateral extent of the circulation layer and the one or more liners may be removed to expose desired portions of the adhesive surface. Accordingly, adhering may entail adhering a dual layer portion of the medical pad to a first patient location and adhering one or more single layer portions of the medical pad to additional patient locations. Such adhering steps may be performed at the same time or at temporally different times. Thermal exchange may occur across the adhesive surface during the first transferring step and during the second transferring step, e.g., without displacing or otherwise repositioning the medical pad relative to the patient.

In some embodiments, the second transferring step comprises fluidly interconnecting the medical pad to a fluid control system. In such embodiments, the second thermal-exchange fluid may be circulated through the circulation layer of the medical pad and the fluid control system.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral following a hyphen to denote one of multiple similar components. When reference is made to a reference numeral without specification to an existing sublabel, it is intended to refer to all such multiple similar components.

FIGS. 1A and 1B illustrates a general configuration for a medical pad in accordance with embodiments of the invention.

FIG. 1C illustrates an alternate configuration of the medical pad of FIG. 1A.

FIGS. 2A and 2B provide top and side views to illustrate a structure for a fluid-circulation layer of the medical pad in an embodiment.

FIG. 2C illustrates an exemplary medical-pad structure for application to a patient in an illustrative embodiment.

FIG. 2D illustrates an alternate exemplary medical-pad structure for application to a patient in an illustrative embodiment.

FIG. 3 illustrates another configuration of a medical pad embodiment.

FIG. 4A is an exploded assembly view of the medical pad embodiment of FIG. 3.

FIG. 4B is a partial cross-sectional view of the medical pad embodiment of FIG. 3.

FIG. 4C is a perspective view of an alternate top layer that may be utilized with the medical pad of FIGS. 3 and 4A.

FIGS. 5A, 5B, and 5C are top views of adjacent layers comprising the medical pad embodiment of FIG. 4A.

FIG. 5D is a bottom view of the layer of the medical pad embodiment of FIG. 3 that is shown in FIG. 5C.

FIGS. 6A and 6B are bottom views of a cut-away, side portion of the layer of the medical pad embodiment of FIG. 3 that is shown in FIGS. 5C and 5D.

FIGS. 7A and 7B comprise a perspective view, and a cross-sectional perspective view, respectively, of an inlet port of the medical pad embodiment shown in FIG. 3 interconnected to a connector of a fluid circulation line illustrated in FIG. 3.

FIGS. 8A and 8B illustrate offset, partial, cross-sectional views of the medical pad embodiment shown in FIG. 3.

FIG. 9 provides a schematic illustration of a plurality of medical pads fluidly interconnected with a fluid control system.

FIG. 10 is a fluid-circuit diagram illustrating on embodiment of a medical pad and related fluid-circulation system in accordance with an embodiment of the invention.

FIG. 11 is a flow diagram summarizing methods of using a medical pad in accordance with embodiments of the invention.

### DETAILED DESCRIPTION

Embodiments of the invention provide a medical pad and methods of contact cooling a patient. The medical pad includes a plurality of layers, at least one of which is a circulation layer for containing a circulatable thermal-exchange fluid that can actively circulate through the layer and at least one of which is a containment layer that encloses a contained thermal-exchange fluid. The combination of an active circulation layer and a passive containment layer in a single medical pad provides a number of benefits in the treatment of conditions where cooling therapy is of value. While medical pads that include an active circulation layer can provide effective cooling, the lack of ready availability of a fluid-control system at the site where the patient is first encountered risks losing time that may be critically important in preventing biological damage that could be mitigated with cooling therapy. The provision of a medical pad with a pre-chilled fluid containment layer allows such a pad to be applied to a patient at the site where patient is first encountered. This allows a patient to receive beneficial cooling earlier in their treatment. In addition, such a medical pad is in place once a fluid control system is available for active cooling (e.g., once a patient is transported to a medical facility). Timing for application of the cooling therapy can be critical in achieving the benefits of the therapy and the combination described herein can decisively make a difference in the level of irreversible biological damage that occurs to the patient, even preventing irreversible damage entirely in some cases. Further, application of a pre-chilled medical pad is more effective than the application of a cool substance, such as ice, for many reasons. For instance, the passive thermal-exchange fluid of the medical pad may be a substance that is better adapted for thermal exchange by having thermal-exchange properties that are more effective. Medical pads that include an adhesive also aid in maintaining a constant position on the patient for application of the cooling therapy. Further, the thermal exchange fluid contained in the fluid containment layer may conform to the shape of an underlying surface (e.g., patient surface) and maintain coverage of and cooling for this underlying surface. That is, the containment layer may limit or prevent the flow of the thermal exchange fluid to lower elevations by use of fluid containment compartments and/or shape holding thermal exchange fluids (e.g., gels).

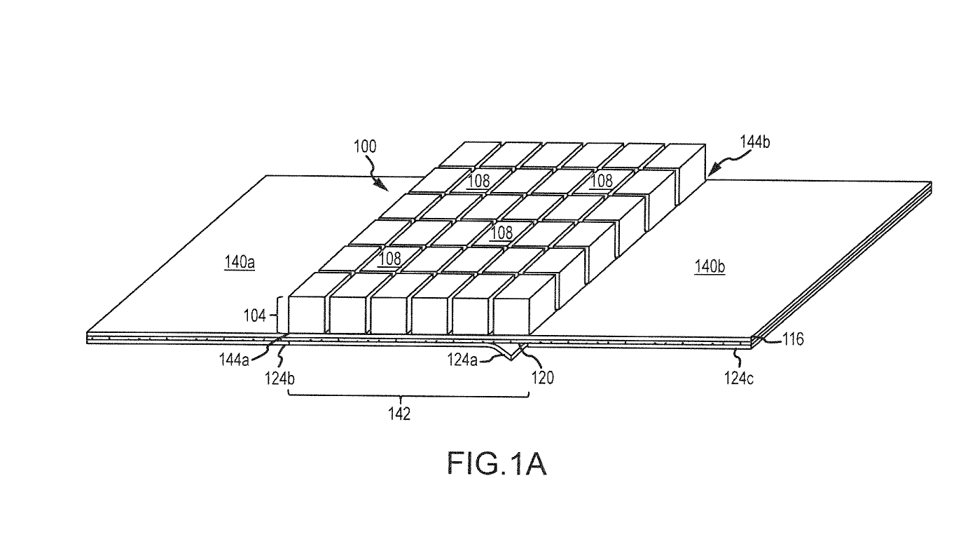

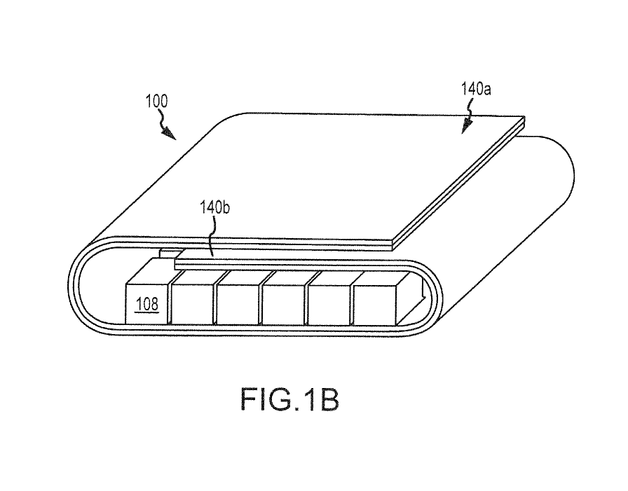

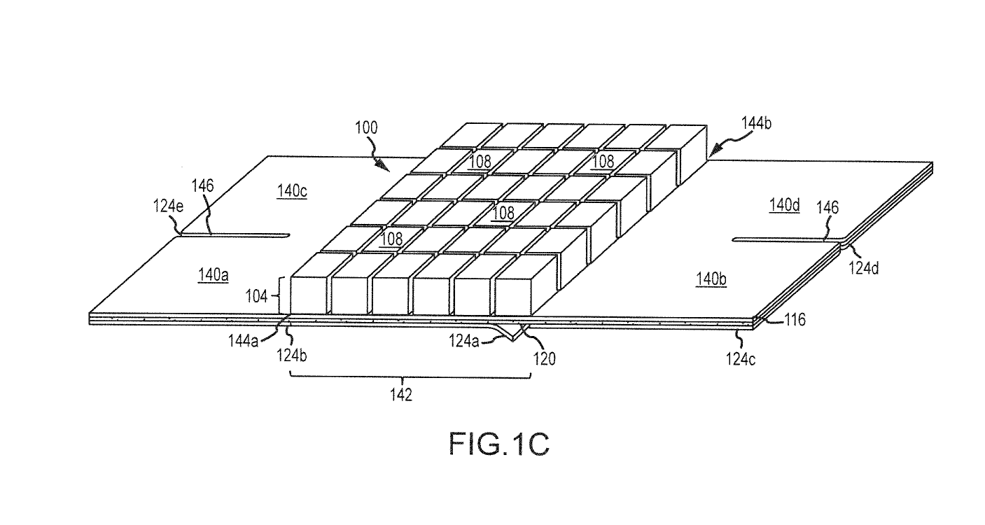

A general overview of one structure for the medical pad according to embodiments of the invention is provided with FIGS. 1A and 1B, which shows a three-dimensional view of a portion of a medical pad 100. A circulation layer 116 comprises a fluid-containing layer for containing the circulatable thermal-exchange fluid that is capable of absorbing and/or releasing thermal energy. The circulation layer 116 may also comprise a conformable thermally conductive layer for facilitating thermal exchange with a patient. Disposed over a portion of a top surface of the circulation layer 116 is a fluid containment layer 104. Specifically, the fluid containment layer 104 may be interconnected with a side or upper surface of the circulation layer 116 that is opposite a skin-contacting side or lower surface of the circulation layer 116.

The fluid containment layer 104 may include a plurality of chambers 108 which may be individually or collectively enclosed in some embodiments, or which may be enclosed in groups in other embodiments. Each of the chambers 108 may be defined by pliable members that project away from the second side of the circulation layer 110 and may have indentations therebetween as illustrated in the drawings. In a specific embodiment in which the chambers 108 are thus provided in a generally rectangular configuration and each have substantially the same size and shape, the containment layer 104 may thus have a waffle-shaped configuration, but this is not a requirement of the invention. In other embodiments, the sizes of the chambers 108 may differ and the chambers 108 may be organized in other than a rectangular configuration, particularly as might be suitable for application to specific portions of the body or for specialized applications. Exemplary medical cooling pads that utilize circulation layers in conjunction with fluid containment layers are illustrated and described in commonly assigned U.S. patent application Ser. Nos. 13/230,663 and 13/662,0256, the entire disclosures of which are incorporated herein by reference for all purposes.

As shown, the fluid containment layer 104 is disposed over less than the entirety of the top side or top surface of the fluid circulation layer 116. In this regard, a first portion 142 of the fluid circulation layer 116 contacts and is attached to the lower or bottom surface of the fluid containment layer 104 to define a dual-layer portion of the medical pad. Remaining portions 140a, 140b of the fluid circulation layer 116 are free of coverage by the fluid containment layer. In this regard, these remaining single-layer portions or ‘flaps’ extend beyond lateral edges of the fluid containment layer 104. In the present embodiment, the first and second flap portions 140a and 140b extend beyond first and second lateral edges 144a, 144b of the fluid containment layer 104. However, it will be appreciated that more or fewer flap portions may be utilized. Further, the term lateral edge is not limited to a straight edge of the fluid containment layer but, rather, denotes any terminal edge of the fluid containment layer 104.

A first thermal-exchange fluid is generally used for circulation through the circulation layer 116 and a second thermal-exchange fluid is generally used for containment in the containment layer 104. As described in further detail below, the first and second thermal-exchange fluids may sometimes be the same fluid, but this is not a requirement of the invention and different thermal-exchange fluids may be used in the circulation and containment layers in different embodiments. The second thermal exchange fluid disposed in the fluid containment layer 104 transfers heat through the skin-contacting side of the fluid circulation layer 116 when the medical pad is applied to a patient surface. That is, the second thermal exchange fluid transfers heat through the fluid circulation layer 116. In order to improve transfer of heat through the fluid circulation layer 104, the fluid circulation layer utilizes an interdigitated structure, as described in further detail below, that allows a significant portion of the second thermal exchange fluid of the fluid containment layer 104 to be in direct contact with the skin-contacting side of the fluid circulation layer. In some embodiments, the second thermal-exchange fluid may comprise a liquid of a gel material, e.g., a shape-holding gel material.

An adhesive surface 120 (see FIG. 1A) may be disposed on the skin-contacting side of the fluid circulation layer 116 for adhering the pad 100 to the skin of a patient. One or more removable liners 124a-c may be provided over the adhesive surface 120 to protect the adhesive surface 120 from contamination while the pad 100 is not in use. The removable liner(s) 124a-c may be selectively removed when the pad 100 is used.

In one approach, the adhesive surface 120 may be provided as a number of downward-facing adhesive strips (e.g., peripheral strips, and/or strips extending across the lateral extent of the medical pad), each having a selectively removable release liner 124a-c disposed thereupon. The adhesive strips may comprise a polyolefin or polyurethane film with hypoallergenic pressure-sensitive acrylate adhesive anchored to the pad 100 with a rubber-based pressure-sensitive adhesive.

In another approach, the adhesive surface 120 may be provided on a conformable, thermally conductive layer. The conformable, thermally conductive layer may comprise a first material, such as a liquid (e.g., water), suspended in a matrix defined by a second material, such as a polymer. In this regard, the liquid may preferably comprise between about 30 to 95 percent by weight of the total weight of the first and second materials. The adhesive surface and thermal transfer layers may be separately comprised of distinct materials. Alternatively, a thermally conductive layer may be comprised of a hydrogel material having sufficient adhesive properties so as to integrally provide the adhesive surface. In such approaches, the adhesive surface 120 may extend across the entirety or at least a majority of the skin-contacting side of medical pad 100 and may be covered by one or more selectively removable release liners 124a-c.

Any embodiment of the medical pad that includes a flap portion that is free of overlying coverage of a fluid containment layer 104 provides additional benefits for the medical pad 100. For instance, the use of a flap portion(s) 140a, 140b, free of an overlying fluid containment layer allows for providing additional fluid circulation layer surface area for actively cooling an increased area of patient tissue when the medical pad 100 is connected to a fluid-control system. In one arrangement, the flap portion(s) have an area or combined area that is at least equal to an area of the dual-layer portion 142 of the fluid circulation layer 116 that is covered by the fluid containment layer. In a further arrangement, this area or combined area is greater than 120%, greater than 140%, greater than 140%, greater than 160%, greater than 180% or greater than 200% of the area of the dual-layer portion 142 of the fluid circulation layer 116 that is covered by the fluid containment layer.

Another benefit of the of the medical pad that includes one or more flap portions that are free of overlying coverage of a fluid containment layer 104 is that prior to connection of a fluid-control system, the flap portions 140a, 140b, may be folded back over the fluid containment layer 104 (see FIG. 1B). This allows the medical pad 100 to be more easily transported to a site where the patient is first encountered. For instance, the pre-chilled medical pad may be folded and disposed in a cooler during transport to the site of a patient. At such time, the pad 100 may be applied to the patient prior to transport back to, for example, a hospital where a fluid-control system may be connected to the fluid circulation layer 116. However, prior to and during such transport, such a patient may receive the benefits of cooling provided by the passive fluid containment layer 104.

As illustrated in FIG. 1B, the flexibility of the flap portions 140a, 140b allows for reducing the size of the medical pad during transportation. Further, such flexibility of the flap portions 140a, 140b in relation to the central portion 142 may improve the adherence of the medical pad to a patient. As shown, the flap portions are operative to pivot relative to the central or dual-layer portion 142 of the medical pad about pivot axes that, in the present embodiment, are generally aligned with the lateral edges 144a, 144b of the fluid containment layer 104. In this regard, it may be noted that these pivot axes allow the flaps to flex between about 70° and 110° above or below the plane defined by the bottom surface of the portion 142 of the medical pad including the overlying fluid containment layer 104. This flexibility of the flap portion allows for applying the medical pad to complex patient surfaces.

In use, it will be appreciated that the second thermal-exchange fluid in the fluid containment layer 104 is typically pre-chilled and in some instances frozen. While the indentations between the individual chambers 108 of the fluid containment layer 104 provide some flexibility when applying the medical pad to an underlying patient surface, the first portion 142 has limited ability to conform to non-planar patient surfaces when the second thermal-exchange fluid is frozen or near frozen. In such an arrangement, the adhesive strips or adhesive surface 120 on the bottom of the first portion 142 of the medical pad may have limited contact with the underlying patient surface and thereby provide limited adhesion to the patient. In contrast, the flap portion(s) 140a and 140b, which are free of the overlying fluid containment layer 104, remain more pliable and can pivot relative to the first portion of the medical pad. In this regard, the flap portions may readily conform to the underlying patient surface. Further, the ability of the flap portions(s) to flex relative to the dual-layer portion 142 allows for more readily attaching the medical pad to portions of a body having smaller surface areas and/or high degrees of curvature. By way of example, the dual-layer portion 142 of the medical pad may be applied to a front surface of a thigh of a patient while the flap portion(s) extend around the side(s) of the thigh. In this situation, the flap portion(s) 104a, 140b, may be utilized to better secure the medical pad to the underlying surface. That is, the flap portion(s) 104a, 140b may be utilized to secure the medical pad in place even if there is limited conformal contact between the underlying patient surface and the dual-layer portion 142 of the medical pad and/or where the underlying patient surface has a high degree of curvature (e.g., around a leg, an arm or torso of the patient).

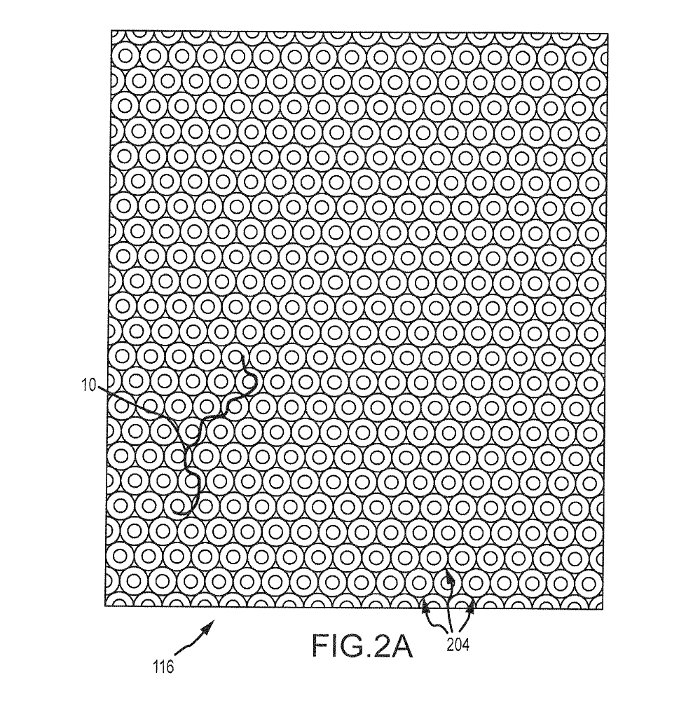

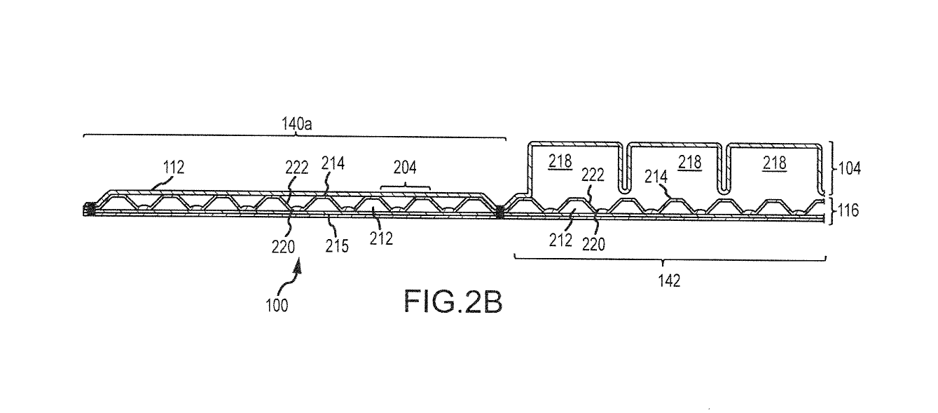

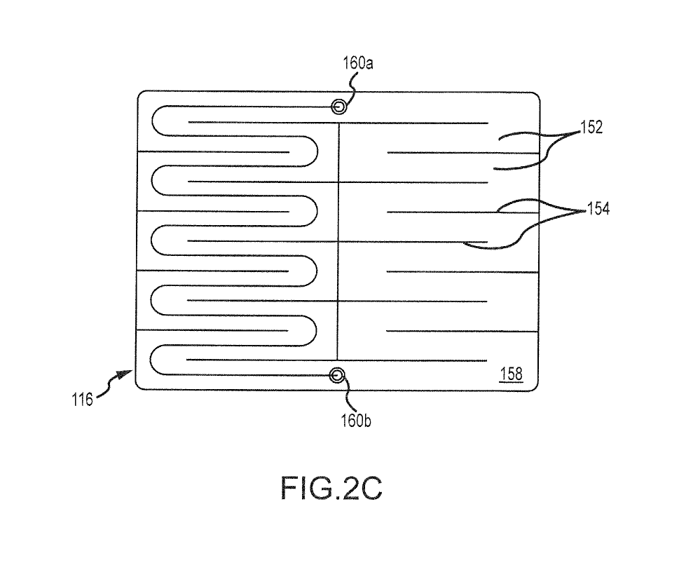

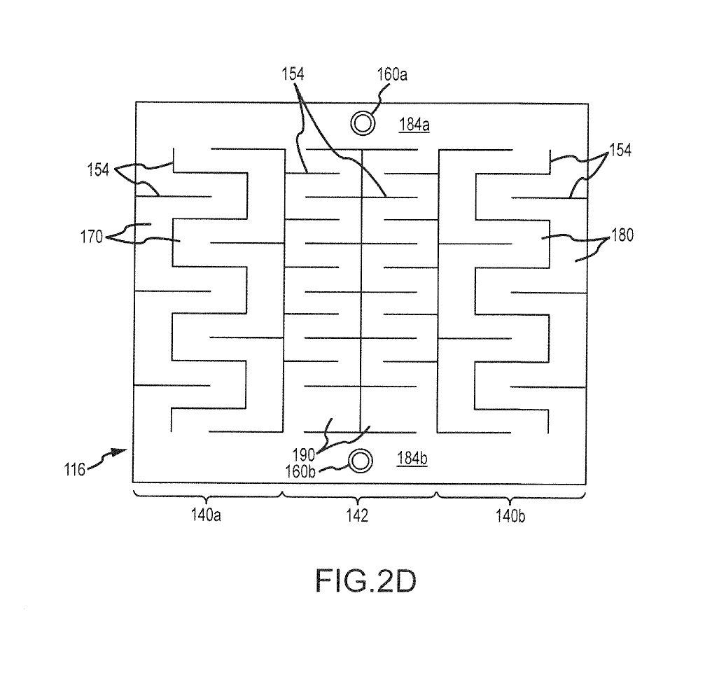

FIGS. 2A and 2B illustrate details of a structure of the circulation layer 116 in an exemplary embodiment with a top view where the fluid containment layer has been removed for purposes of illustration and a side view, respectively. The circulation layer 116 comprises a dimple-matrix having a plurality of dimples 204 structured to achieve a desired level of thermal communication with the thermal-exchange fluids in the circulation and containment layers. Fluid paths are provided within the circulation layer 116 by channels 212 formed by the structure of the circulation layer 116 between the dimples 204. This allows the first thermal-exchange fluid to flow in meandering, or tortuous, pathways around the dimples. The availability of multiple meandering paths advantageously allows the first thermal-exchange fluid to flow through the circulation layer 116 with wide coverage, enhancing thermal exchange with the patient's skin and increasing effectiveness of the cooling. An example of a portion of one potential path is illustrated with bold line 210.

The cross-sectional view of FIG. 2B illustrates more particularly how the structure of the dimple matrix defines the channels 212 in one particular embodiment, and how thermal exchange with both the first and second thermal-exchange fluids is achieved. Specifically, a structure 214 (e.g., comprising a polymer-based material) may define the dimple matrix with channels 212 sealably provided between the structure 214 (e.g., top surface of fluid circulation layer) and a sheet-like layer 215 (e.g., bottom surface of fluid circulation layer). Typically, the structure 214 is formed of a sheet layer of a non-permeable material (e.g., polymer-based material). Formed into the structure 214 are a plurality of depressions 222 and corresponding projections 220 that form the dimples 204. In the present embodiment, the depressions 222 are formed into a top surface of the structure 214 and thereby define corresponding projections 220 on a bottom surface of the structure 214. Accordingly, when the sheet-like layer 215 is disposed over the bottom surface of the structure 214, a top surface of the sheet-like layer 215 is juxtaposed against the projections 220. Thus, the spaces between the projections 220 and the sheet-like layer 215 define the channels 212 and the circulation layer 116. Thermal exchange occurs between the first thermal-exchange fluid and a patient's skin at locations defined by the channels 212 (e.g., spaces between the projections 220 and sheet-like layer 215) where the first thermal-exchange fluid is disposed adjacent to, and thereby in direct or near-direct thermal communication with the skin of the patient when the medical pad is applied.

Thermal exchange between the second thermal-exchange fluid and the patient's skin may occur between the channels 212, at those locations where structure 214 of the circulation layer 116 allows for the second thermal-exchange fluid to fill the depressions 222 of the dimples 204. That is, the second thermal-exchange fluid fills the depressions 222 over the portion 142 of the fluid circulation layer covered by the fluid containment layer 104. In the illustrated embodiment, separate enclosed chambers 218 comprising the containment layer 104 may be defined over one or more dimples 216 to provide adjacent positioning and direct or near-direct thermal communication between the skin of the patient and the second thermal-exchange fluid in the containment layer 104. Like the structure 214, the containment layer 104 is formed is formed of a sheet layer (e.g., upper sheet layer) of a non-permeable material (e.g., polymer-based material) that is molded or otherwise formed to a desired shape and disposed over the upper surface of the structure 214 to define the individual chambers 218 of the containment layer. In various embodiments overlying chambers 218 may be sized to each extend over a plurality of dimples 204 to define separate chambers for containing the second thermal-exchange fluid. In a further arrangement, the portion of the structure 214 that is not covered by the portion of the containment layer defining the chambers 218, may be covered by an insulative layer 112. This insulative layer 112 may reduce thermal exchange through the top surface of the flap portions of the medical pad when applied to a patient. The insulative layer 112 may be formed of a sheet layer of non-permeable material and may be separatively formed or be formed as part of the containment layer. In the latter regard, it will be appreciated that spaces between the insulative layer 112 and the underlying structure 214 are free of the second thermal exchange fluid.

With the illustrated structure, approximately 50% of the skin-contacting side of the circulation layer 116 is provided adjacent to and thereby in direct or near-direct thermal communication with the circulation layer and approximately 50% of the skin-contacting side of the circulation layer 116 is provided adjacent to and thereby in direct or near-direct thermal communication with the containment layer 104. That is, the total area of the bottom surfaces of the depressions 222 may contact 50% of the sheet-like layer 215. Thus, 50% of the sheet-like layer may be in thermal contact with the first thermal exchange fluid that passes through the circulation layer 116. Likewise 50% of the sheet-like layer 215 of the portion 142 of the fluid circulation layer 116 covered by the fluid containment layer 104 may be in thermal contact with the second thermal exchange fluid contained within the containment layer 104. The structure may be varied in other embodiments to achieve different relative levels of thermal communication between the different layers and/or for different portions of the layers. For example, in varying embodiments, greater than 20%, greater than 30%, greater than 40%, greater than 50%, greater than 60%, greater than 70%, or greater than 80% of the skin-contacting side of the circulation layer 116 is provided in direct or near-direct thermal communication with the first thermal-exchange fluid. In other embodiments, greater than 20%, greater than 30%, greater than 40%, greater than 50%, greater than 60%, greater than 70%, or greater than 80% of the skin-contacting side of the portion 142 of the circulation layer 116 covered by the fluid containment layer 104 is provided in direct or near-direct thermal communication with the second thermal-exchange fluid.

It is noted that while the embodiment illustrated in FIGS. 2A and 2B generally provides approximately 100% of the skin-contacting side of the portion 142 of the circulation layer 116 covered by the fluid containment layer 104 be in communication with one or the other of the thermal-exchange fluids, this is also not a specific requirement of the invention. The total area of the skin-contacting side of the circulation layer 116 may at times have less than 100% of its area in communication with one of the thermal exchange layers (e.g., flap portions 140a, 140b may have less than 100% of their area in contact with the fluid circulation layer). While there may be advantages in treating certain conditions to having 100% of the area in communication with a thermal-exchange fluid purely for treatment reasons, the many varying shapes of parts of the body where treatment may be applied may make it preferable to have configurations in which less than 100% of the area is in thermal communication in order to provide greater structural integrity to the medical pad for such applications, to configure specialized circulation paths for certain areas of the body, or for other reasons such as may be evident to those of skill in the art. In specific embodiments, greater than 50%, greater than 60%, greater than 70%, greater than 80%, or greater than 90% of the area of the skin-contacting side of the circulation layer 116 is provided in thermal communication with one or both of the first and second thermal-exchange fluids. The level of thermal communication with the different thermal-exchange fluids may also be provided as desired with different configurations of the containment layer.

In the embodiment of FIGS. 1A-2B, fluid in the containment layer 104 is provided in direct thermal communication with the circulation layer 116 so that the depressions in the top of the structure of the circulation layer may hold some of the second thermal-exchange fluid. This embodiment also allows the different chambers 108 of the containment layer 104 to be in fluidic communication with each other. In an alternative embodiment, each chamber might be enclosed individually or may be enclosed in groups so that fluid communication is provided among separate sets of chambers. Such embodiments may be suitable for certain specialized applications in which different thermal properties are desired at different positions of the medical pad.

FIG. 2C provides one illustration of a medical pad configuration of the bottom surface of the fluid circulation layer 116, with the patient-facing layer thereof removed. As will be appreciated by those of skill in the art, there are many configurations that may be used depending on such factors as the part or parts of the body to which the medical pad is to be applied, the nature of the condition to be treated, the environment where the condition is to be treated, i.e., whether it is in a hospital, physician's office, accident site, or otherwise.

The configuration includes areas 158 where dimples of the circulation layer (not shown) may be provided, e.g., as described above. Channels 152 may be defined by ribs 154, or raised portions. Fluid is circulated through the circulation layer 116 through fluid ports that may be provided at manifold bonding sites 160a, 160b to provide access to the channels 152 within the circulation layer. The location, configuration, and orientation of the ports may be selectively established to provide various advantages. In particular, the ports may be provided to avoid patient weight from creating localized high-pressure areas on the skin by pressing the port or attached tubing against the skin of the patient. Reducing such high-pressure areas reduces the risk of causing pressure ulcers. Also, the tubing can exit off an a patient support platform (e.g., an emergency liter) without multiple turns, thereby reducing the risk of interconnected tubing buckling or kinking, which would limit fluid flow.

The ribs 154 prevent the first thermal-exchange fluid from following a path directly between the input and output ports of the circulation layer, e.g., going directly from site 160a to site 160b. Instead, the first thermal-exchange fluid flows along a path such as illustrated with bold line 164. It is noted that this exemplary path is schematic; at a more detailed level, the actual paths followed by the first thermal-exchange fluid are meandering paths as dictated by the dimple structure of the layer and as explained above in connection with FIGS. 2A and 2B.

In further embodiments, it may be desirable to alter the configuration of the channels in the different portions of the fluid circulation layer. FIG. 2D provides another illustration of a medical pad configuration of the bottom surface of the fluid circulation layer 116, with the patient-facing layer thereof removed. The dimples of the circulation layer are not shown for purposes of illustration.

In this embodiment, the fluid circulation layer 116 may include rib members 154 that define a first pair of adjacent fluid channels 170 in the first flap portion 140a, a second pair of adjacent fluid channels 180 in the second flap portion 140b, and a third pair of adjacent fluid channels 190 in the central portion 142 all of which extend between the fluid ports 160a, 160b of the pad 10. As may be appreciated, fluid may be circulated from port 160a to port 160b, or alternately from port 160b to port 160a.

In the illustrated embodiment, the first and second pairs of adjacent of channels 170, 180 are of coincidental, serpentine configuration. More particularly, each of the channels comprising the first and second pairs of adjacent of channels 170, 180 is of an S-shaped configuration. Further, such channel of each pair of channels 170, 180 may be of a substantially common length, e.g., within about 25% of an average length as measured along their respective center paths and may also have a substantially common average width, e.g., within about 25% of an average of their average widths.

The third pair of channels 190 are also disposed in a mirror configuration. As illustrated, each of the channels comprising the third pair of channels 190 follows a serpentine path. Further, it should be noted that this pair of channels 190 may be of a substantially common length, e.g., within about 25% of an average length as measured along their respective center paths and may also have a substantially common average width, e.g., within about 25% of an average of their average widths.

Fluid staging chambers 184a, 184b are provided at the fluid ports 160a, 160b, respectively. Such staging chambers serve to distribute fluid and normalize fluid flow through the plurality of channels 170, 180 and 190.

In order to accommodate the flap potions, while also providing for effective thermal exchange with a patient, the configurations and relative widths of the first plurality of channels 170 and second plurality of channels 180, should be further addressed. In particular, the first and second plurality of channels 170, 180 each include a U-shaped portion extending through the flap portions 140a, 140b. In the resulting medical pad, the pivot axes of the various flap portions are substantially parallel to the bases of the U-shaped portions of channels 170, 180 as well as the outer side edges of the flap portions.

Specific configurations for the fluid channels may be as described in, for example, U.S. Pat. No. 6,648,905, the entire disclosure of which is incorporated herein by reference for all purposes. For instance, a first plurality of channels within the circulation layer may be of coincidental, serpentine configuration. More particularly, each of the channels comprising the first plurality of channels may be of a generally S-shaped configuration. Such channels may be of a substantially common length, such as in embodiments where each channel has a length within about 15% of an average length as measured along their respective center paths.

Similarly, the channels may also have a substantially common average width, such as in embodiments where each channel has a width within about 25% of an average of the average widths of each channel. A second plurality of channels may also be disposed in a coincidental manner and similarly have substantially common lengths and widths as defined. The structure may also include fluid staging chambers at the fluid ports to distribute fluid and normalize fluid flow through the different pluralities of channels.

Variation of the medical pad is possible and considered within the scope of the presented inventions. For instance, it will be appreciated that each portion of the medical pad which is free of the overlying fluid containment layer may be divided into two or more independent sub-flaps. As illustrated in FIG. 1C, the medical pad again includes a dual layer portion 142 having a fluid containment layer 104 overlaying a fluid circulation layer 116. Disposed on either side of the dual layer portion 142 are first and second flap portions where the fluid circulation layer 116 is free of the overlying fluid containment layer 104. In this embodiment, the pad includes a plurality of flap portions 140a, 140b, 140c and 140d where each pair of flaps 140a, 140c and 140b, 140d is disposed on opposing sides of the portion 142 of the pad including the overlying fluid containment layer 104. Each pair of flaps 140a, 140c and 140b, 140d are separated by slits 146 to allow each individual flap 140a, 140b, 140c and 140d pivotable, conformable positioning about a patient. That is, these slits 146 allow for the separate manipulation of each of the flap portion independent of the positioning of an adjacent flap. Further, it should be noted that the side flap portions may be separately attached or left unattached to a patient during a medical procedure. In this regard, the first portion 142 of the medical pad and each flap 140a, 140b, 140c and 140d may include a separate removable liner 124a-e that covers its adhesive bottom surface.

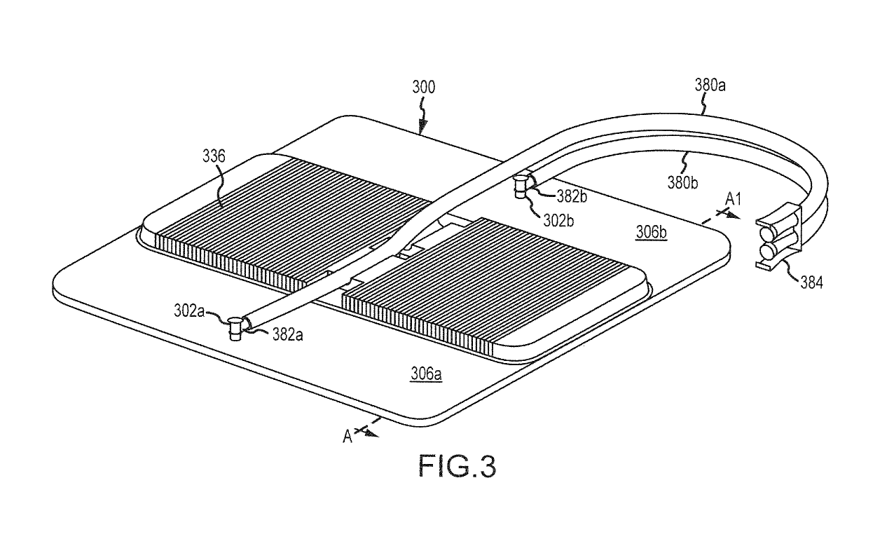

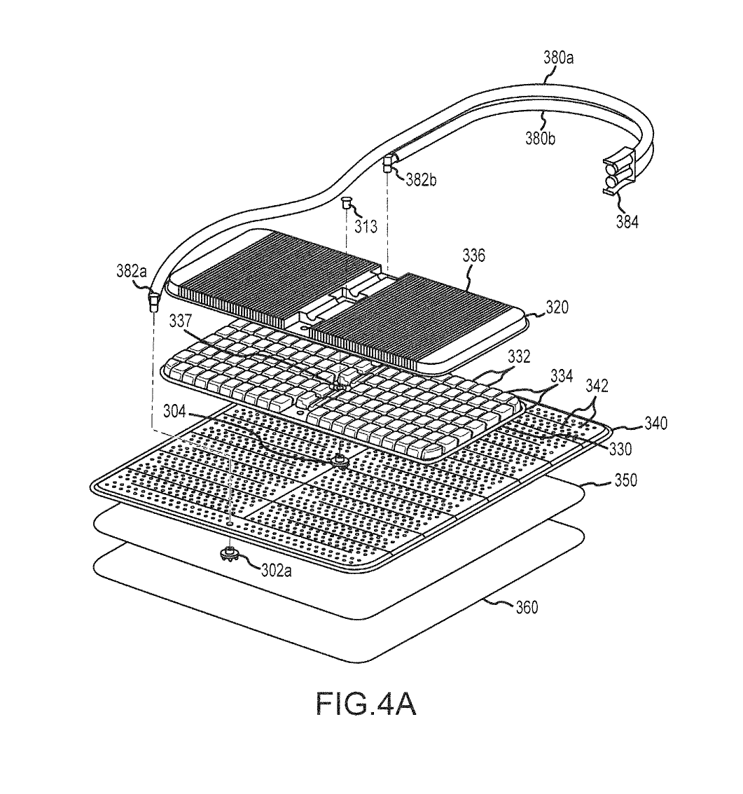

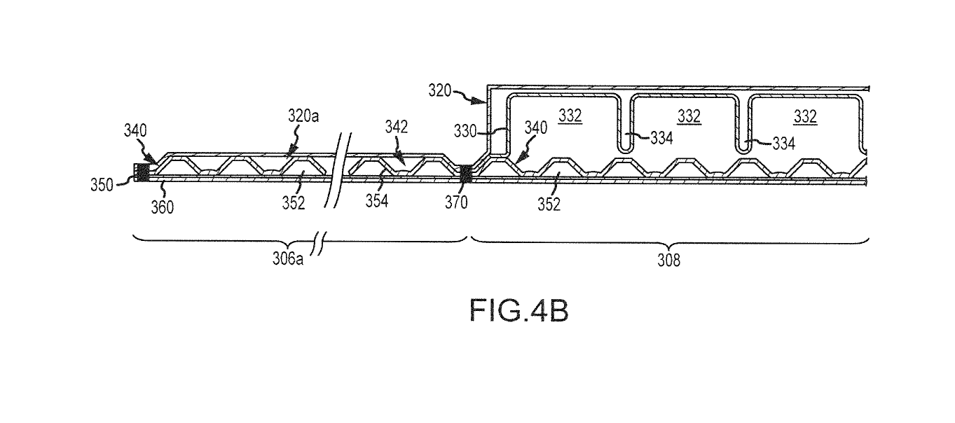

FIGS. 3-8B variously illustrate a more detailed embodiment of a medical pad 300. As shown in FIGS. 3 and 4A, fluid circulation lines 380a, 380b may be provided to circulate a first fluid thermal-exchange through the medical pad 300. For example, a connector 382a provided at a first end of fluid circulation line 380a may be fluidly interconnectable to a fluid inlet port 302a in a first flap 306a of the fluid circulation layer of the medical pad 300, and a connector 382b provided at a first end of fluid circulation line 380b may be fluidly interconnectable to a fluid outlet port 302b in a second flap 306b of fluid circulation layer of the medical pad 300. Second ends of fluid circulation lines 380a, 380b may be provided for selective interconnection to and disconnection from a fluid control system discussed herein below. In the illustrated embodiment, a connector device 384 may be provided at the second ends of the first and second fluid circulation lines 380a, 380b for interconnection with the fluid control system. In one approach, connector device 384 may be of a type described in U.S. Pat. No. 6,827,728.

Reference is now made to FIGS. 4A and 4B which further illustrate an exploded view of FIG. 3 and a partial cross-sectioned view of FIG. 3 taken along section lines A-A′. In the illustrated embodiment, medical pad 300 may include a top layer 320, a containment layer 330, an intermediate layer 340, an interface layer 350, and a bottom layer 360. As illustrated, the various layers may be arranged in a stacked, or laminate, fashion. Various additional configurations are envisioned, including configurations designed for specific anatomic regions of use.

The intermediate layer 340 and interface layer 350 may be provided to define a circulation layer (e.g., channels 352; see FIG. 4B) therebetween, wherein a first thermal-exchange fluid may flow into and out of such circulation layer via fluid circulation lines 380a, 380b. Further, the intermediate layer 340 and containment layer 330 may be provided to define a fluid containment layer therebetween for containing a second thermal-exchange fluid disposed in, for example, one or more chambers 332. FIG. 4B, also illustrates a sheet-like interface layer 350 having a top surface disposed adjacent to the bottom surface of the intermediate layer 340.

As will be appreciated, a second thermal-exchange fluid contained in the containment layer may be provided to cool a patient, independent from and/or in overlapping relation with the circulation of a first thermal-exchange fluid through a central portion 308 of the fluid circulation layer. Further, a first thermal-exchange fluid may be circulated through the flap portions 306a, 306b fluid circulation layer to cool a patient, independent from and/or in conjunction with the patient cooling by a second thermal-exchange fluid contained within the containment layer overlapping the central portion 308 of the circulation layer.

In one approach, adjacent ones of the top layer 320, containment layer 330 and the intermediate layer 340 may be interconnected (e.g., via R1 welding of copolymer materials comprising such layers at a joint or seam 370 extending generally about the peripheries of the top layer 320 and containment layer 330. The interface layer 350 may be connected across a top side thereof to a bottom side of intermediate layer 340. The interface layer 350 may define an adhesive surface or layer. In one approach, the patient interface layer 350 may comprise a hydrogel material disposed across the lateral extent of the bottom side of the intermediate layer 340 (e.g., across all or substantially all of the bottom side of the medical pad). For example, hydrogel materials may be utilized that comprise a polymer/water matrix marketed by AquaMed Technologies of Langhorne, Pa., U.S.A. The patient interface layer 350 may further include a removable liner or bottom layer 360 that may be readily removed from the adhesive surface of the interface layer 350 at the time of placement of medical pad 300 on a given patient for contact cooling (e.g., direct adhesive engagement with the skin of a patient). The adhesive surface may display a peel value at initial skin application of about 20 g/in. to 80 g/in. to facilitate fixed positioning on a patient, yet facilitate removal after use.

As illustrated in FIGS. 4A and 4B, the containment layer 330 may comprise a plurality of chambers 332 that project upward and away from a bottom side of the containment layer and upward from a top side of the intermediate layer 340, with indentations 334 between such chambers 332. A top side of the intermediate layer 340 may be provided with a plurality of depressions 342, e.g., a dimple-matrix, extending across the lateral extent thereof. Correspondingly, a bottom side of the intermediate layer may be provided with a plurality of projections 354. Collectively, the spaces between the projections 354 on the bottom surface of the intermediate layer 340 and the top surface of the interface layer 350 define fluid flow channels 352 of the circulation layer. In one approach, the chambers 332 and depressions 342 may be disposed in opposed, face-to-face relation for fluid communication therebetween. In this regard, at least a portion of a second thermal-exchange fluid contained by the containment layer overlying the central portion 308 of the pad may be contained by the plurality of depressions 342 and the plurality of chambers 332 defining the containment layer.

In one approach, the chambers 332 and indentations 334 may be arranged in rows and columns to facilitate flexure of the medical pad along the indentations 334 for conformal engagement of medical pad 300 with a patient. In this regard, each of the layers 320, 330, 340 and 350 may be of a pliable construction to facilitate curvature, or flexure, along the lateral and/or longitudinal dimensions thereof. By way of example, each of the layers may comprise a copolymer material such as a polyolefin material (e.g., ethylene-vinyl acetate).

To further facilitate conformal positioning of medical pad 300 and/or enhanced thermal transfer between a patient and a first thermal-exchange fluid circulated through the circulation layer, the depressions 342 may be arranged in staggered rows and columns. In this regard, the depressions 342 on the top side of intermediate layer 340 provide corresponding projections on the bottom side of intermediate layer 340. In turn, tortuous flow paths around the projections may be defined within the fluid circulation layer.

Top layer 320 may be provided to define an insulative layer, or air space, between the top layer 320 and containment layer 330. In this regard, such an insulative layer may surround chambers 332 to enhance thermal exchange between the second thermal-exchange fluid and a patient during use. That is, the insulative top layer 320 provides a pocket of trapped air that acts to insulate the upper surface of the chambers 332 in the containment layer 330. An additional portion 320a of the insulative top layer may extend over the flap portion 306 of the medical pad. Further, to enhance flexibility of the top layer 320, a series of corrugations 336 may extend across the width of the top layer 320. See FIGS. 3 and 4A. Such corrugations 336 allow the top layer to stretch and compress to facilitate flexure of the underlying chambers 332 when the medical pad 300 is applied to a non-planar surface.

In another embodiment illustrated in FIG. 4C, the top layer 320 includes a plurality of indentations or recesses 338 that extend across a lateral extent thereof. As shown, these recesses are significantly deeper as measured from a top surface of the top layer 320 as compared to the corrugations 336 as illustrated in FIG. 4A. Accordingly, these recesses 338 permit greater flexibility of the top layer 420 and, hence, the underlying layers of the medical pad 300. In order to provide recesses 338 having such an increased depth, it is in some instances necessary that the top layer recesses be positioned between indentations 334 between the chambers 332 of the underlying fluid containment layer 330.

In the illustrated embodiment, the top layer recesses 338 extend across a lateral width of the top layer in an accordion-shaped configuration. These recesses allow the top layer to expand and collapse. However, it will be appreciated that the top layer may incorporate recesses in other configurations such as across its lateral length in instead of and/or in addition to the recesses across its lateral width. That is, the top layer recesses 338 may define a waffle-shaped configuration similar to the indentations 334 between the chambers 332 in the fluid containment layer. Such an arrangement may allow the top layer to expand and/or collapse in two or more directions. Further, the number and/or spacing of top layer recesses 338 may be varied. For instance, while the top layer recesses 338 may be disposed in indentations between every other row of chambers in the fluid containment layer, may be disposed between every row of chambers, every third row etc. Likewise, if the top layer recesses are provided between the columns of chambers 332, the number and/or spacing of such indentations may also be varied. Further, the number and/or spacing of the recesses may be varied independently across the lateral width and lateral length of the medical pad. For instance and by way of example only, the recesses may be disposed between every other set of adjacent chamber rows and between every third set of adjacent chamber columns.

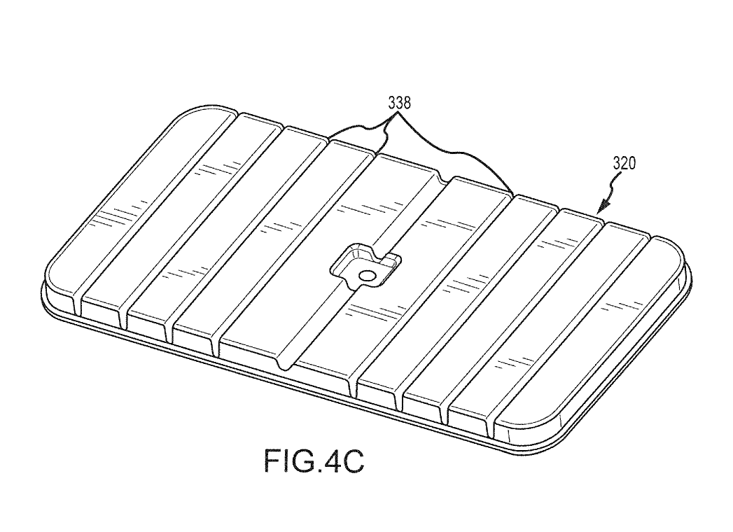

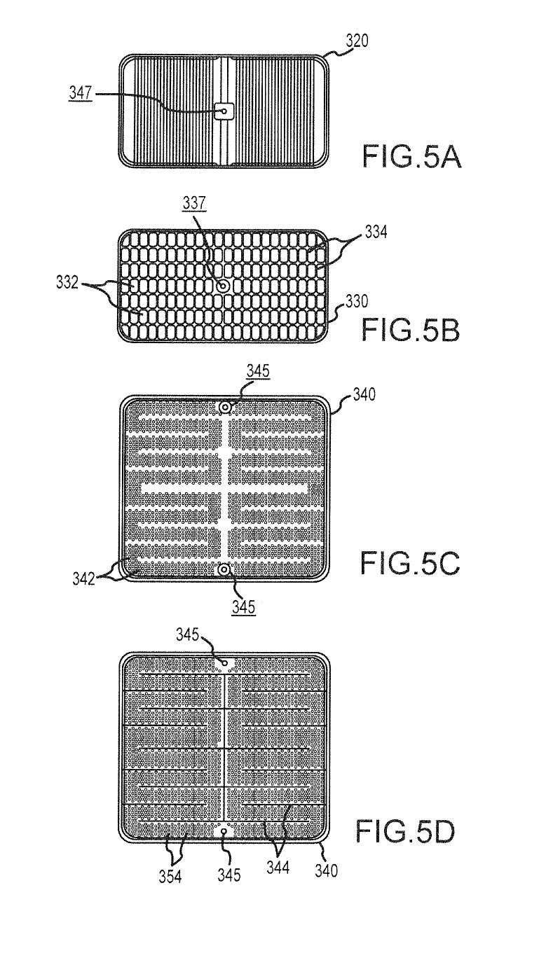

In relation to the above noted features, reference is now also made to FIGS. 5A, 5B and 5C, which illustrate top views of layers 320, 330 and 340, and FIG. 5D which illustrates a bottom view of layer 340. Top layer 320 may define widthwise pleats or corrugations, different ones of which may be positionable between columns of chambers 332 of containment layer 330.

Further, top layer 320 may include one or more opening(s) 347 for receipt of a fill port 304 therethrough, as shown in FIG. 4A, for selective use in flowing a second thermal-exchange fluid into the containment layer (e.g., during assembly of medical pad 300).

Reference is now made to FIG. 5B which illustrates containment layer 330 with chambers 332 and indentations 334 defining a matrix of rows and columns. Additionally, containment layer 330 may include one or more opening(s) 337 for receipt of fill port 304 therethrough, as shown in FIG. 4A.

As illustrated in FIGS. 5C and 5D, intermediate layer 340 also includes openings 845 for positioning inlet port 302a and outlet port 302b therethrough. In relation to FIG. 5C, depressions 342 are shown on the top side of the intermediate layer 340. In relation to FIG. 5D, such depressions 342 define downward projections 354 on the bottom side of the intermediate layer 340. Additionally, ribs 344 are provided that project downward on the bottom side of the intermediate layer 340. In turn, tortuous flow paths may be defined for the flow of a first thermal-exchange fluid between ribs 344, around the projections 354 defined by depressions 342. As may be appreciated, such tortuous fluid flow may occur between inlet port 302a and outlet port 302b.

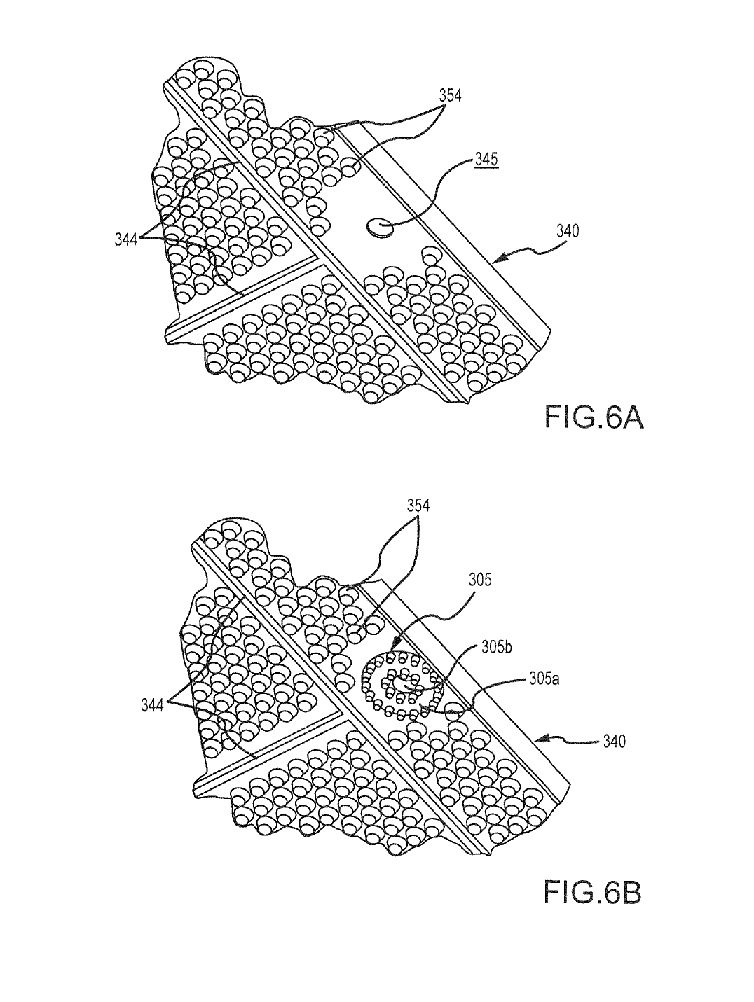

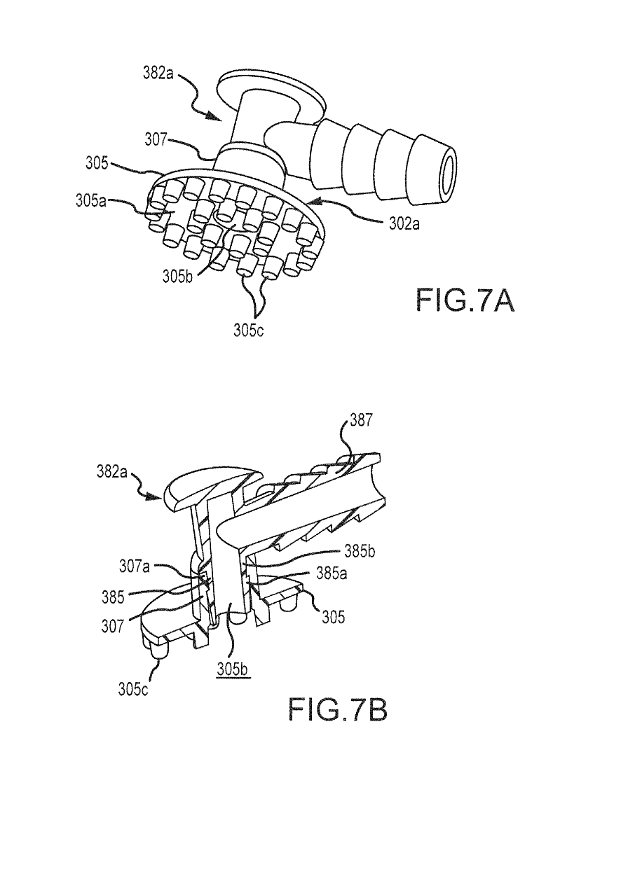

FIGS. 6A and 6B, and FIGS. 7A and 7B variously illustrate the interconnection of fluid ports with the fluid circulation layer. FIG. 6A illustrates a cutaway portion of a bottom side a side edge portion of intermediate layer 340, showing an opening 345 extending therethrough, and illustrating projections, corresponding with projections 354 and ribs 344 projecting downward on the bottom side of the intermediate layer 340. As shown, projections 354 are of a frusto-conical configuration.

FIG. 6B illustrates the cutaway portion shown in FIG. 6A with an enlarged end 305 of inlet port 302a disposed on a bottom side of intermediate layer 340. As shown, enlarged end 305 includes a disk portion 305a, an aperture 305b and stand-off members 305c projecting away from disk portion 305a about aperture 305b. Inlet port 302a may be of a sufficiently rigid construction (e.g., comprising an integral, molded plastic material), such that stand-off members 305c maintain a desired layer-to-layer spacing for fluid flow at aperture 305b.

Inlet port 302a is shown interconnected to a connector 382a in FIGS. 7A and 7B. As illustrated, in addition to the enlarged end 305, inlet port 302a comprises a tubular portion 307 in fluid communication with aperture 305b. As may be appreciated, tubular portion 307 may be sized to fit through openings 345 of the intermediate layer 340. Further, tubular portion 307 may be configured for selective interconnection with connector 382a.

For example, and as shown in FIG. 7B, tubular portion 307 may be configured together with connector 382a for one-way, snap-fit interconnection. For such purposes, a top end of tubular portion 307 may be sized to receive a tubular port 385 at connector 382a. Further, tubular portion may be provided with an inwardly protruding lip 307a. In turn, first tubular port 385 may have a tapered end portion 385a and adjacent recess for snap-fit receipt of the lip 307a of the tubular portion 307 of the inlet port 302a. As further shown in FIGS. 7A and 7B, connector 382a may be of an L-shaped configuration that includes first and second tubular ports 385 and 387, adjoined at elbow 386, thereby yielding a low-profile interconnection footprint. Tubular part 387 may be barbed for retentive, fluid-type interconnection with tubing comprising fluid circulation line 380a. As may be appreciated, outlet port 302b and connector 382b may be configured in a manner analogous to inlet port 302a and connector 882a described above, respectively.

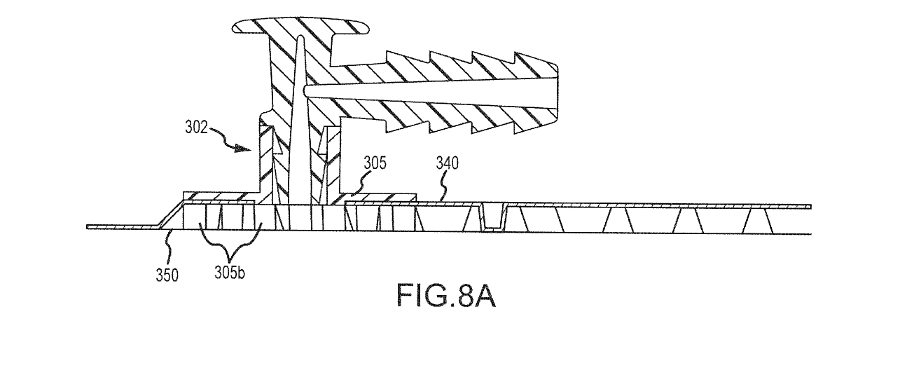

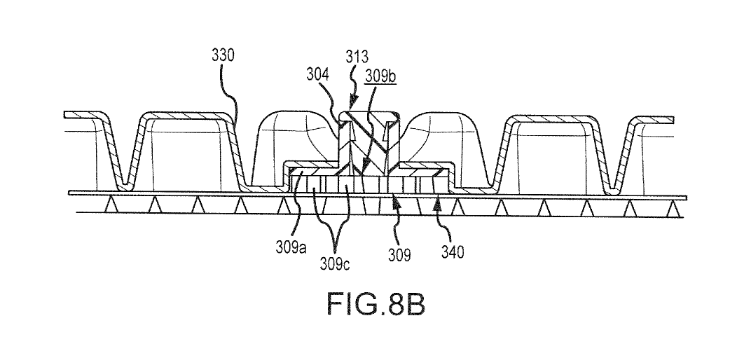

Reference is now made to FIG. 8A which illustrates a cross-sectional views of medical pad 300, with inlet port 302a interconnected to connector 382a. Fluid circulation lines 380a is not shown to facilitate discussion. As shown in FIG. 8A, the enlarged end 305 of inlet port 302a is positioned between fluid interface layer 350 and intermediate layer 340 to provide for a first thermal-exchange fluid flow in to and out of the circulation layer defined by interface layer 350 and intermediate layer 340. As noted, the stand-off members 305b maintain a minimum desired spacing to facilitate fluid flow in to and out of the fluid circulation layer.

As further shown by FIG. 8B, fill port 304 may comprise and enlarged end 309 disposed between a bottom side of containment layer 330 and the top side of intermediate layer 340. The enlarged end 309 may include a disk portion 309a, an aperture 309b and stand-off members 309c projecting away from disk portion 309a about aperture 309b. The stand-off members 309c maintain a minimum desired spacing to facilitate fluid flow into the containment layer. The fill port 304 further includes tubular portion for selective fluid interconnection to and disconnection from a source of the second thermal-exchange fluid during filling of the containment layer. A plug 313 may be provided to close-off tubular portion after filling of the containment layer.

As may be appreciated, medical pad 300 may be readily assembled and readied for use. For example, interface layer 350 may be provided with a removable layer 360 removably attached to the bottom adhesive surface of the fluid interface layer. In turn, the top side of the interface layer 350 may be interconnected to a bottom side of the intermediate layer 340 with enlarged ends 305 of ports 302a and 302b positioned therebetween, and tubular portions 307 located through openings 345. Such interconnection may occur subsequent to or prior to interconnection of the top layer 320, containment layer 330, and intermediate layer 340. As may be appreciated, the enlarged end 309 of fill port 304 may be disposed between intermediate layer 340 and containment layer, with tubular portion positioned through openings 347, and 337, prior to such interconnection.

In contemplated arrangements, after filling the fluid containment layer with the second thermal-exchange fluid, the medical pad 300 may be cooled. By way of example, in some embodiments, medical pad may simply be disposed in a freezer, yielding the medical pad 300 ready for use.

At the time of use, bottom layer 360 may be removed from an adhesive surface on all or a portion (e.g., control portion 308) of the bottom side of the fluid interface layer 350, and the adhesive surface of medical pad 300 may be contacted with a patient to initiate patient cooling. As may be appreciated, such patient cooling provides for thermal exchange between the second thermal-exchange fluid and the patient. Such thermal exchange may occur, for example, during transport of a patient.

Further, when patient cooling is desired via thermal exchange between a first thermal-exchange fluid circulated through medical pad 300 and a patient, connectors 382a, 382b of fluid circulation lines 380a, 380b may be interconnected to ports 302a, 302b, and connector 384 may be interconnected to a fluid circulation control system, wherein the first thermal-exchange fluid may be circulated through circulation layer of medical pad 300 to achieve patient cooling in tandem with or independent from patient cooling via the second thermal-exchange fluid (e.g., during and after the second thermal-exchange fluid warms). Further in conjunction with circulation through the circulation layer one or all flap portions 306 may be adhered to the patient if such flap portions have not previously been adhered.

A number of different thermal-exchange fluids may be used in different embodiments of the invention for both the first and the second thermal-exchange fluids, including gases and liquids such as water. As will be appreciated by those of skill in the art, the thermal-exchange characteristics of the pad 100 or 300 may depend on the thermal properties of the thermal-exchange fluids that are used. In particular, some embodiments make use of thermal-exchange fluids that include impurities, which may be in solid, liquid, or gaseous form, to tailor the thermal-exchange properties of the pad.

Table I indicates the thermal properties and densities of certain exemplary materials that may be used in different embodiments and of the thermal properties and densities of biological tissues that may interact thermally with the pad 100.

TABLE I

| | Heat Capacity | Thermal conductivity | Density |
|---|---|---|---|
| Material | (kJ/kg ° C.) | (W/mK) | (g/cm³) |
| Aluminum | 0.9 | 230 | 2.71 |
| Graphite | 0.7 | 170-370 | 2.2 |
| Copper | 0.38 | 390 | 8.97 |
| Water | 4.186 | 0.57 | 1 |
| Ice | 2.1 | 1.7 | |
| Muscle tissue | 3.6 | 0.36-0.5 | 1 |
| Bone | 1.2 | 0.5 | 2 |
| Fat | 1.67 | 0.186-0.3 | 0.93 |
| Blood | 4 | 0.472-0.62 | 1 |

As noted in the table, a combination of water and a metal or other material such as those listed in the table may yield a greater thermal conductivity. If water is supplemented, for example, with 10 vol. % aluminum or graphite, its thermal conductivity increases by a factor of about 20. By mixing the substances in this way, the fluidic properties of water may advantageously be used while simultaneously increasing thermal conductivity. Although aluminum and graphite have similar thermal-conductivity, the specific-heat capacity of graphite offers additional advantages over the use of aluminum in some embodiments.

In one embodiment, a first thermal-exchange fluid may comprise a liquid such as water for circulation through the circulation layer. Further, the second thermal-exchange fluid may comprise liquid of a gel material. In one approach, a cellulose gel material may be utilized that is flowable into the containment layer and curable to assume a shape-holding state within the containment layer. For example, a carboxmethyl cellulose (CMC) gel may be utilized that includes aluminum acetate to crosslink the CMC and form a shape-holding gel.

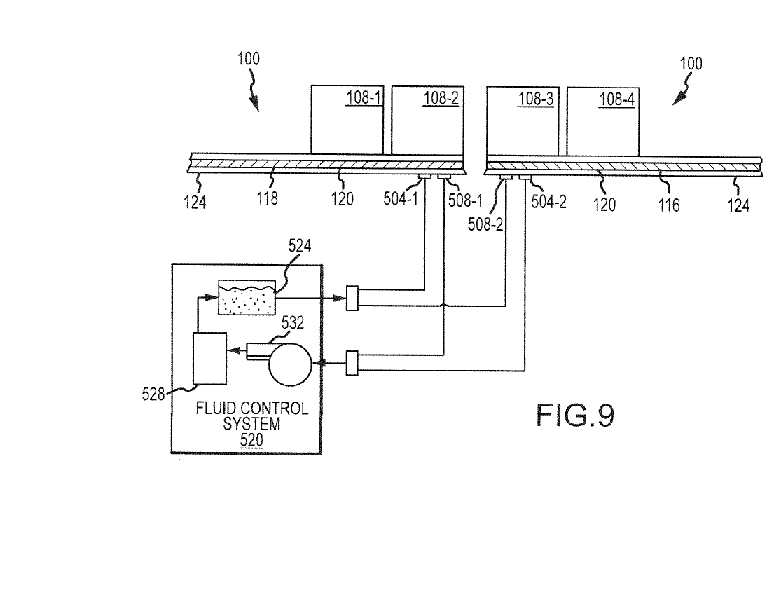

FIG. 9 provides a schematic illustration of how circulation may be achieved through the circulation layer 116. The following discussion utilizes reference numbers associated with the embodiment shown in FIGS. 1-2B, however, it will be appreciated that the discussion is equally applicable to the medical pad 300 of FIGS. 3-8B. The drawing shows a plurality of pads, such as may be appropriate for a configuration to be applied to various parts of the body where the shape of the body makes it less effective to use a single pad. For instance, application to the torso may involve the use of a pad 100 for the right side of the patient and a pad 100 for the front side of the body where it curves. Application to the legs might involve separate pads for each of the legs, etc. Each of the plurality of pads 100 is shown to have the same general structure as the pad 100 described in detail in connection with FIG. 1, including both a circulation layer 116 and a containment layer 104 having a plurality of chambers 108.

Fluid may be circulated through the fluid ports 504 and 508 by an interconnectable fluid-control system module 520, such as through interconnected tubing lines. In one arrangement, the fluid-control system module 520 comprises a pump 532 for drawing fluid through the pads 100 under negative pressure, usually less than about −10 psi, although other pressures may be used in different embodiments. At least one thermal-exchange device 528 is provided for cooling the circulated fluid and a fluid reservoir 524.

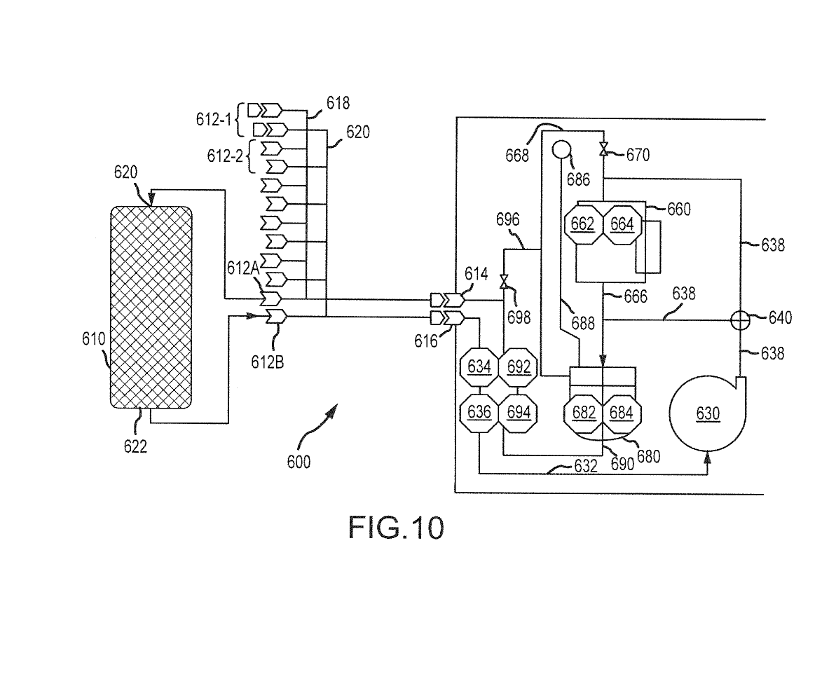

A fluidic circuit diagram is shown in FIG. 10 to illustrate in greater detail how fluid is circulated through the medical pad, designated by reference number 610 in the drawing. The medical pad is connected with the fluid circulating system 600 using pad-connector pairs 612. Each pad-connector pair 612 includes an inlet connector 612A for connection with an inlet 620 of the medical pad 610 and an outlet connector 612B for connection with an outlet 622 of the medical pad 610. Both the inlet and outlet connections may be made with flexible tubing or similar structure suitable for fluidic connection. Merely by way of illustration, the embodiment shown includes six pad-connector pairs 612 to permit connection of six medical pads 610 with the fluid circulating system 600. But it should be appreciated that the invention is not limited by the number of pad-connector pairs 612 and that different embodiments might have a greater or lesser number of pad-connector pairs 612. Each inlet connector 612A of the pad-connector pairs 612 is connected via an inlet feeder line 618 to a main inlet connector 614, and each outlet connector 612B of the pad-connector pairs 612 is connected via an outlet feeder line 620 to a main outlet connector 616. The fluid circulating system 600 also includes a pump 630, a temperature storage module 660, and a fluid reservoir 680.

The pump 630 is connected downstream via a pump inlet line 632 from the main outlet connector 616 and is preferably self-priming. A temperature sensor 634 and a pressure sensor 636 in the pump inlet line 632 measure the temperature and pressure respectively of the fluid exiting the pad 610 or pads connected with the fluid circulating system 600. Information from the pressure sensor 636 may be used in controlling the speed of the pump 630 so that generally constant negative pressure is maintained. The pump 630 is connected upstream via pump outlet lines 638 and a three-way valve 640 with both the reservoir 680 and the temperature storage module 660.

The temperature storage module 660 includes cooling elements 662 and a temperature sensor 664. The cooling elements 662 may be activated to cool fluid within the temperature storage module 660 to a desired temperature detectable by the temperature sensor 664. The temperature storage module 660 is connected via a primary temperature storage module outlet line 666 upstream from the reservoir 680 so that fluid that has been cooled to a desired temperature within the temperature storage module 660 flows therefrom to the reservoir 680 while the pump 630 is operating, i.e., pumping fluid therethrough. The three-way valve 640 may be regulated to control the proportion of fluid that flows to the reservoir 680 directly from the pump 630 and the portion of fluid that flows from the pump 630 through the temperature storage module 660 to the reservoir 680 in order to control the temperature of the fluid flowing into the pad 610. The temperature storage module 660 is also connected via a secondary temperature storage module outlet line 668 to the reservoir 680. A normally open valve 670 in the secondary temperature storage module outlet line 668 permits fluid to drain from the temperature storage module 660 to the reservoir 680 when the pump 630 is not operating.

The fluid reservoir 680 includes a level sensor 682 for detecting a level of fluid within the reservoir 680 and cooling element 684 for precooling fluid within the reservoir 680. When desirable, such as when the level sensor 682 indicates that the fluid level has fallen below a specified level, additional fluid may be added to the reservoir through a fill port 686 that is connected with the reservoir 680 by a fill line 688. Preferably, the reservoir 680 has a nonmixing inlet and outlet in order to minimize undesirable temperature variations of fluid within the reservoir. The outlet of the reservoir 680 is connected via a reservoir outlet line 690 to the main inlet connector 614. A temperature sensor 692 and a flow sensor 694 may be provided in the reservoir outline 690. The temperature sensor 692 measures the temperature of fluid provided to the pad inlets via the inlet feeder line 618. Information from the temperature sensor 692 may be used in regulating the three-way valve 640 to control the fluid temperature. Information from the flow sensor 694 and the temperature sensor 634 in the pump inlet line 632 may be used in determining the heat transfer between the patient and pads connected to the fluid circulating system 600. A drain line 696 with a normally closed two-way valve 698 is provided for draining the pads to the reservoir 680 when the cooling procedure is complete.

Other configurations may be used for the fluid circulating system 600 in alternative embodiments, examples of which are illustrated and described in commonly assigned U.S. Pat. No. 6,197,045, the entire disclosure of which is incorporated herein by reference for all purposes.

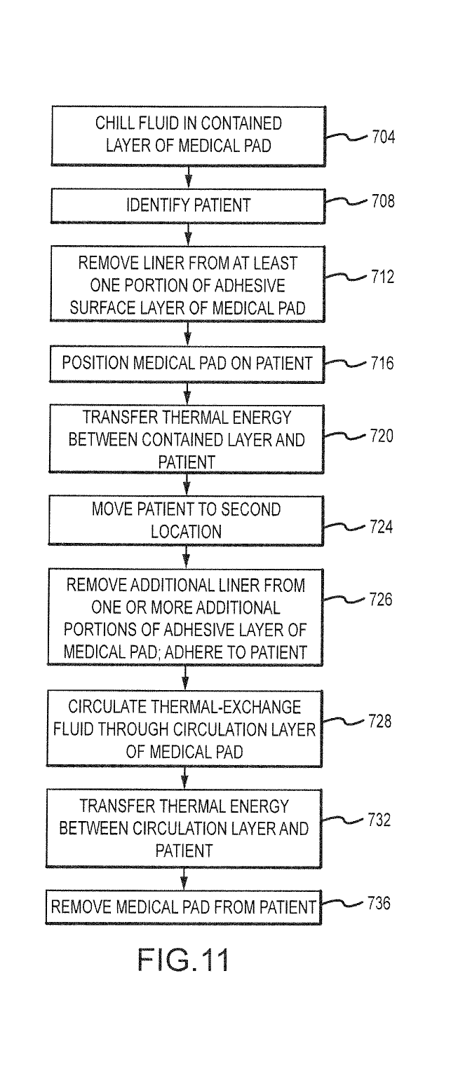

FIG. 11 provides a flow diagram that illustrates methods for using a medical pad in accordance with embodiments of the invention. While the flow diagram sets for specific functions that are performed and illustrates them in an exemplary order, these are not intended to be limiting. In various alternative embodiments, some of the functions may be omitted, others not specifically illustrated may additionally be performed, and/or the order may be changed from that illustrated specifically in the drawing.

The method begins at block 704 by chilling the second thermal-exchange fluid in the containment layer of the medical pad. As previously noted, different thermal-exchange fluids may be used in different embodiments and therefore the phase-transition points of the fluid may differ in different embodiments. In some embodiments, the second thermal-exchange fluid has a freezing point equal to or less than 0° C. In those embodiments where the second thermal-exchange fluid comprises water mixed with another substance, the freezing point may be higher or lower than 0° C. In certain embodiments, the second thermal-exchange fluid may comprise a liquid such as water comprising a shape-holding gel material that may be chilled to 0° C. or less, such that the liquid is in a frozen state or at least a partially frozen state at block 704, and wherein the shape-holding gel maintains an initial configuration as the second thermal-exchange fluid warms during use.

It is also noted that chilling the second thermal-exchange fluid at block 704 may or may not involve a phase change in the fluid. For example, if the second thermal-exchange fluid is pure water, it may be chilled to a temperature on either side of its freezing point of 0° C. without deviating from the intended scope of the invention. Indeed, even if the second thermal-exchange fluid is frozen as part of the chilling at block 704, it is still considered to be a “fluid” as the term is used herein. Further, if the second thermal-exchange fluid has an evaporation point that is crossed as part of the chilling at block 704 so that it changes phase from a gas to a liquid, it is still considered to be a “fluid” as the term is used herein.

Use of the medical pad is generally expected to result in the transfer of thermal energy to the second thermal-exchange fluid, and such transfer may result in reversal of a phase change that occurs as part of the chilling at block 704. Such embodiments are also specifically intended to be within the scope of the invention.

At block 708, a patient is identified who is expected to benefit from application of a cooling therapy. The patient may be suffering from a stroke, head trauma, or other injury or disease that may be effectively treated with cooling therapy. It is specifically noted, though, that it is not a requirement of the invention that the patient be suffering from any type of disorder, whether it be an injury-caused disorder or otherwise. In some embodiments, the cooling therapy may be used as an adjunct to the application of other medical procedures, such as where a patient undergoing surgery is identified as likely to benefit from the application of cooling therapy.

The medical pad is applied to the identified patient at blocks 712 by removing a liner or plurality of liners from the adhesive layer of one or more portions of the medical pad, depending on whether the embodiments use a generally continuous adhesive layer and release liner or have a plurality of adhesive strips and corresponding plurality of release liners. In embodiments where no adhesive is used, block 712 may be omitted. At block 716, the medical pad is positioned on the patient. It is generally expected that the pad will be placed in contact with skin tissue with the adhesive being used to adhere the portion of the pad below the fluid containment layer to the skin and thereby generally maintain its position on the patient during the cooling therapy. Additionally other portions (e.g., flap portions) free of an overlying fluid containment layer may be adhered to the skin of the patient. But in alternative embodiments, the pad may be positioned on other types of tissue, although such embodiments may omit the use of an adhesive.

The nature of the medical pad as described above, particularly its thermal properties, allows a transfer of thermal energy between the contained layer and the patient at block 720. The transfer results in cooling of the patient, at least locally in the area where the pad is applied and with consequent heating of the second thermal-exchange fluid.

At block 724, the patient is moved to a second location where the first thermal-exchange fluid may be circulated through the circulation layer of the medical pad at block 728. If necessary, additional liners may be removed from additional portions of the pad to adhere these additional portions of the pad to the patient as shown in optional block 726. This results in thermal energy being transferred between the circulation layer and the patient at block 732. To realize fluid circulation, the medical pad may be selectively interconnected to a fluid control system. Circulation of the first thermal-exchange fluid may be achieved using the fluid control system as described in connection with FIGS. 5 and 6 and as also described in commonly assigned U.S. Pat. Nos. 6,197,045, 6,648,905, and 6,799,063, all of which are incorporated herein by reference in their entireties.

Movement of the patient at block 724 may take place in a number of different ways that reflect a variety of implementations of the invention. Such movement also combines with other aspects of the invention, particularly including the use of two thermal-exchange fluids that are used differently, to achieve numerous benefits. For example, there may be circumstances in which an appropriate fluid-control system is not available at the location where the medical pad is applied to the patient at block 716. This may occur, for instance, in emergency settings where a medical pad of the type described herein is maintained in an ambulatory vehicle for access by paramedics who do not have access to the fluid-control system at the emergency site. It may also occur in settings where a physician maintains medical pads of the type described herein at his or her office, but where the fluid-control system is maintained at a hospital. Still other settings where such circumstances may exist include clinics or nurses' offices in schools, which might maintain medical pads for use, but which lack the larger and more specialized fluid-control system equipment. Once the treatment has been applied, the medical pad may be removed from the patient at block 736. In conjunction with such removal, the medical pad may be disconnected from the fluid control system and disposed of.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

Having described several embodiments, it will be recognized by those of skill in the art that various modifications, alternative constructions, and equivalents may be used without departing from the spirit of the invention. Accordingly, the above description should not be taken as limiting the scope of the invention, which is defined in the following claims.

1. A medical pad comprising:
a fluid circulation layer having a first surface and a second surface with at least one fluid path between the first and second surfaces for containing a first thermal-exchange fluid circulatable therethrough;
a fluid containment layer disposed on the second surface of the fluid circulation layer and extending over a portion of less than all of the second surface of the fluid circulation layer, wherein the fluid containment layer encloses a second thermal-exchange fluid.

2. The medical pad of claim 1, wherein at least a first portion of the fluid circulation layer extends beyond a lateral edge of the fluid containment layer.

3. The medical pad of claim 2, wherein at least first and second portions of the fluid circulation layer extend beyond first and second lateral edges of the fluid containment layer.

4. The medical pad of claim 2, wherein said first portion of the fluid circulation layer extending beyond a lateral edge of the fluid containment layer is pivotally interconnected to a portion of the fluid circulation layer covered by the fluid containment layer.

5. The medical pad of claim 4, wherein said first portion is pivotally connected along a pivot axis.

6. The medical pad of claim 2, further comprising:
an adhesive disposed on the first surface of the fluid circulation layer, wherein the adhesive is adapted for releasable adhesive contact with skin of a patient.

7. The medical pad of claim 6, further comprising:
a removable release liner disposed over the adhesive.

8. The medical pad of claim 6, further comprising:
a first release liner disposed over adhesive applied to a portion of the first surface of the fluid circulation layer adjacent to a portion of the second surface covered by the fluid containment layer; and
a second release liner disposed over the adhesive applied to the first portion of the first surface of the fluid circulation layer that extends beyond the lateral edge of the fluid containment layer, wherein the first and second release liners are removable without removal of the other of the first and second release liners.

9. The medical pad of claim 6, wherein the adhesive comprises a hydrogel material.

10. The medical pad of claim 1, wherein the second thermal exchange fluid of the containment layer is in thermal contact with a portion of the first surface of the fluid circulation layer.

11. The medical pad of claim 10, wherein the fluid circulation layer further comprises a plurality of depressions formed in the second surface, wherein the second thermal exchange fluid is disposed in at least a portion of the depressions.

12. The medical pad of claim 11, wherein bottom surfaces of the depressions contact the first surface of the fluid circulation layer.

13. The medical pad of claim 11, wherein a total area of bottom surfaces of the depressions comprise at least 30% of an area of the portion of the second surface of the fluid circulation layer covered by the fluid containment layer.

14. The medical pad of claim 11, wherein a total area of bottom surfaces of the depressions comprise at least 50% of an area of the portion of the second surface of the fluid circulation layer covered by the fluid containment layer.

15. The medical pad of claim 1, wherein the fluid containment layer comprises a plurality of chambers.

16. The medical pad of claim 15, wherein the plurality of chambers comprise a plurality of enclosed chambers each enclosing a corresponding different portion of the second thermal-exchange fluid therewithin.

17. The medical pad of claim 16, wherein each of the plurality of chambers projects away from the second surface of the fluid circulation layer with indentations therebetween.

18. The medical pad of claim 17, wherein the plurality of chambers define a waffle-shaped configuration.

19. The medical pad of claim 15, further comprising:
an insulative layer extending over the plurality of chambers to insulate the fluid containment layer.

20. The medical pad of claim 1, further comprising:
a first port fluidly interconnected to the fluid circulation layer for circulating the first thermal-exchange fluid into the fluid circulation layer; and
a second port fluidly interconnected to the fluid circulation layer for circulating the first thermal-exchange fluid out of the fluid circulation layer.

21. The medical pad of claim 1, wherein the second thermal-exchange fluid comprises a liquid in a gel material.

22. The medical pad of claim 21, wherein the gel material is shape-holding.

23. The medical pad of claim 1, wherein at least one of the first thermal-exchange fluid or the second thermal-exchange fluid has a thermal conductivity that exceeds 5.0 W/mK.

24. The medical pad of claim 1, wherein the fluid containment layer further comprises;
a port for disposing the second thermal-exchange fluid into the fluid containment layer.

25. The medical pad of claim 1, wherein the second thermal-exchange fluid has a freezing point of 0° of less.

26. The medical pad of claim 25, wherein the at least one of the first thermal-exchange fluid or the second thermal-exchange fluid comprises a liquid containing a material having a thermal conductivity that exceeds a thermal conductivity of the liquid by at least a factor of 100.

27. The medical pad of claim 1, wherein the fluid circulation layer further comprises:
a plurality of fluid paths between the first and second surfaces.

28.-65. (canceled)
